(19) United States
(12) Patent Application Publication
(10) Pub. No.: US 2012/0029554 A1
(43) Pub. Date: Feb. 2, 2012

(54) MEDICAL HAND TOOL

(76) Inventor: Winfried KREIDLER, Tuttlingen (DE)

(21) Appl. No.: 13/192,809

(22) Filed: Jul. 28, 2011

(30) Foreign Application Priority Data
Jul. 29, 2010 (DE) DE 202010010843.8

(51) Int. Cl. A61B 17/28

(52) U.S. Cl. 606/205

(57) ABSTRACT

A medical hand tool (1) has a first and a second hand lever (2, 3), which have a handle (4, 5) and a tool part (6, 7) each, and which are pivotably connected to one another via a pivot pin (10). The first hand lever (2) forms a flat first base plate (8) in the area between the handle (4) and the tool (6) and the second hand lever (3) forms a flat second base plate (9) in the area between the handle (5) and the tool (7), in the area of which the pivot pin (10) is arranged. In order to be able to clean such a hand tool (1) reliably and completely by a machine, e.g., in a liquid-jet-generating cleaning machine, provisions are made for the pivot pin (10) to be provided in an end area passing through second base plate (9) with a radially expanded ring flange (12), which has a distance from the first base plate (8) that corresponds to at least twice the thickness of the second base plate (9) mounted on the pivot pin (10). The second hand lever (3) can be moved along the pivot pin (10) into a position, in which the contact surfaces (17, 18) of the flat base plates (8, 9), which face one another, are exposed.

### CROSS REFERENCE TO RELATED APPLICATIONS

This application claims the benefit of priority under 35 U.S.C. §119 of German Utility Model DE 20 2010 010 843.8 filed Jul. 29, 2010, the entire contents of which are incorporated herein by reference.

### FIELD OF THE INVENTION

The present invention pertains to a scissor-like or forceps-like medical hand tool with a first and a second hand lever, which have a handle and a tool part each and which are pivotably connected to one another via a pivot pin, whereby the first hand lever forms a flat first base plate in the area between its handle and its tool and the second hand lever forms a flat second base plate in the area between its handle and its tool, in the area of which the pivot pin is arranged, whereby the pivot pin is arranged fixed in the first base plate and pivotably accommodates the second base plate provided with a bearing bore.

### BACKGROUND OF THE INVENTION

In hand tools of this type, the two base plates of the two hand levers lie flat on top of one another in the normal operating state. The result of this is that many places, especially in the area of the pivot pin connecting the two hand levers or base plates to one another cannot be completely and reliably cleaned by means of a machine. These areas about the pivot pin of the base plates lying flat on top of one another are not directly accessible for jets of the cleaning liquid in a cleaning machine but rather are covered by other parts.

### SUMMARY OF THE INVENTION

The underlying object of the present invention is to provide a hand tool of the type mentioned in the introduction, such that it can be cleaned reliably and completely by means of a machine, e.g., in a liquid-jet-generating cleaning machine, i.e., that no “dead spaces” form, into which the cleaning jets cannot directly penetrate.

This object is accomplished according to the present invention in that the pivot pin passes through the second base plate and is provided in its end area passing through the second base plate with a radially expanded ring flange, which has a distance from the first base plate that corresponds to twice the thickness of the second base plate mounted on the pivot pin or is greater than twice the thickness of the second base plate, such that the second hand lever can be moved in a maximum pivoting position to the first hand lever along the pivot pin into a position, in which the contact surfaces of the flat base plates, which face one another, are exposed.

Such a designed hand tool can be brought into an opening position, in which the normally critical areas about the pivot pin are exposed, such that these can also be detected by the cleaning jets and be reliably cleaned. For this, the two hand levers can be brought into an “open” pivoting position, in which it is possible to adjust or to move the two base plates in relation to one another in the direction of the pivot pin, without the hand tool being able to fall apart. For this, the pivot pin is connected, on one side, in a fixed manner to the first base plate of the first hand lever. The second base plate of the second hand lever is pivotably accommodated via its bearing bore on the pivot pin in relation to the first base plate or hand lever. To guarantee that the second base plate cannot be removed from the pivot pin in the open pivoting position or that it does not slide off same inadvertently, the pivot pin has at its free end a radially protruding ring flange, which is used as a type of stop during the moving of the second base plate along the pivot pin. To guarantee that the two base plates have sufficient distance from one another for accessibility of the cleaning liquid, the distance between the first base plate, which has the fixed pivot pin, and the ring flange is selected to be at least twice as great as the thickness of the second base plate.

An embodiment may be provided to guarantee an as sufficient moving path as possible, but to prevent an interfering “projecting length” of the pivot pin in case of the base plates lying on top of one another in their operating position. According to this, provisions are made for the bearing bore of the second base plate of the second hand lever mounted on the pivot pin to be provided on the outside with a radially expanded hole expansion accommodating the ring flange of the pivot pint. If the second base plate of the second hand lever is pushed along the pivot pin, then the ring flange reaches the area of this hole expansion, in which the ring flange is partly or entirely accommodated in case of maximum “moving path.” With this embodiment, it is possible to keep the pivot pin short, such that, when using the hand tool, the pivot pin does not project too far from the second base plate of the second hand lever.

The embodiment results in the technical handling advantage of being able to lock the two hand levers in their maximum pivoting position opposite one another, so that they retain this pivoting position even during the cleaning process in the cleaning machine. According to this, provisions are made for the first hand lever to have, in the area of its tool, a stop face on the outside protruding in the direction of the pivot pin and lying in the pivoting area of the second base plate of the second hand lever, and for the second base plate to be able to be supported with an edge area at the stop face in its position raised from the first base plate.

The embodiments according to the invention facilitate the mutual locking of the two hand levers in their maximum pivoting position.

Thus, provisions are made for the stop face to lie in the area of a plane, in which the inner contact surface, which faces the first base plate, of the second base plate of the second hand lever, is located, when the second base plate is located on the pivot pin in its position removed farthest from the first base plate of the first hand lever.

Furthermore, for this, provisions are made for the stop face to be provided with an oblique guiding edge.

Provisions may be made for the base plates to have an at least approximately congruent surface shape and to have essentially the same thickness. Due to this congruent shaping, the base plates form a uniform outer contour, especially in the closed state of the hand levers.

Furthermore, a circular ring web, protruding in the area surrounding the hole expansion in the direction of the pivot pin, may be provided, in which the hole expansion continues. This ring web ends in the area of the ring flange of the pivot pin in case of base plates lying on top of one another. Due to this embodiment, the pivot pin with its ring flange is accommodated in the hole expansion at least partly even in the closed pivoting position of the two hand levers, with the base plates lying on top of one another. Thus, the risk of injury, for example, due to “pinching” of a finger of an operator during the moving of the second base plate along the pivot pin is considerably reduced.

The present invention is explained in detail below based on the attached drawings. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a perspective view of a forceps-like medical hand tool in the closed state;

FIG. 2 is an enlarged perspective partial view of the hand tool from FIG. 1 with hand levers located in their open pivoting position with base plates “still” lying on top of one another;

FIG. 3 is the partial view from FIG. 2 with the base plate of the hand lever “moved” along the pivot pin;

FIG. 4 is a partial section of the hand tool from FIG. 1 through the pivot pin with the hand tool closed;

FIG. 5 is the partial section from FIG. 4 with hand levers located in their open pivoting position and base plates “pushed” away from one another;

FIG. 6 is a partial top view of the hand tool from FIG. 1 which is in contact with a holding device in its open pivoting position, whereby the holding device is shown in section VI-VI from FIG. 7;

FIG. 7 is a lateral view VII from FIG. 6 of the hand tool together with the suspension means;

FIG. 8 is a perspective partial view of a hand tool with a ring web on the outside of the second base plate protruding in the area surrounding the pivot pin; and

FIG. 9 is a partial section of the hand tool from FIG. 8 through the pivot pin with the hand tool closed.

### DESCRIPTION OF THE PREFERRED EMBODIMENTS

Referring to the drawings in particular, as is apparent from the drawings, the hand tool 1 in the exemplary embodiment shown is a type of tweezers. However, the present invention is not limited to this type of “hand tool” but rather extends to other hand tools, which have two hand levers pivotable in relation to one another. Thus, the hand tool 1 could also be designed, for example, as scissors with two blades or as pincers with two transversely running cutting edges. Other embodiments for other applications are also conceivable.

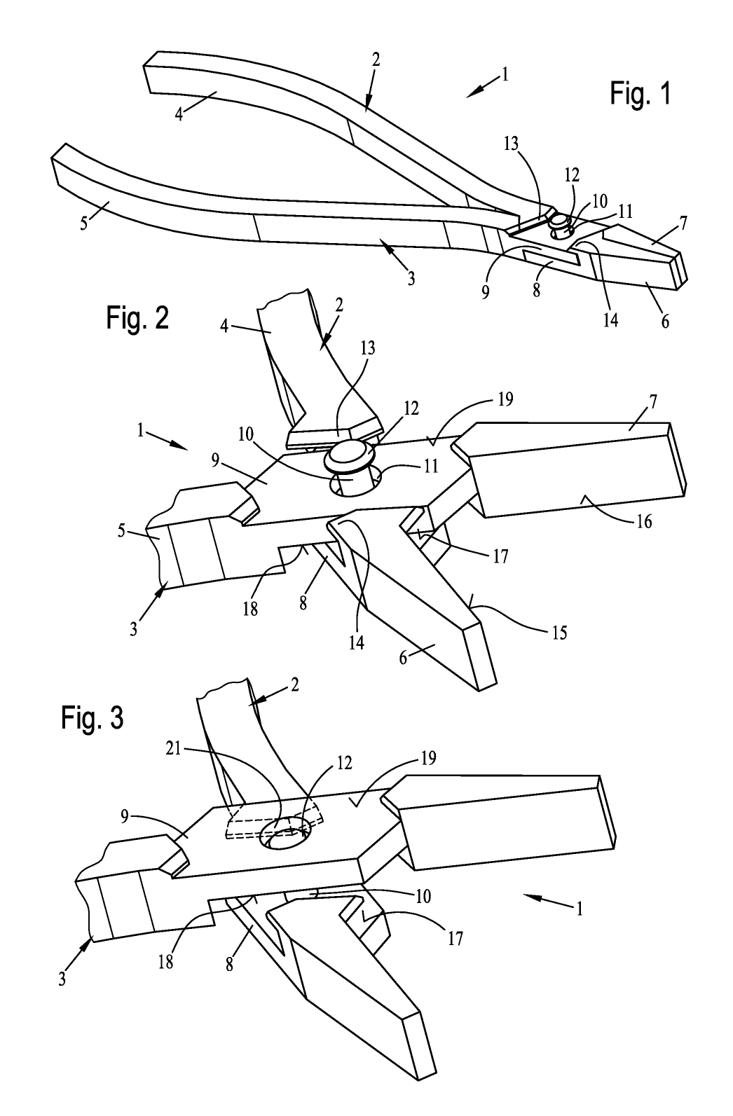

The hand tool 1 shown isometrically in FIG. 1 consists of a first hand lever 2 and a second hand lever 3. The first hand lever 2 is provided with a first handle 4 and second hand lever 3 is provided with a second handle 5. A first or second tool each in the form of a first or second clamping jaw 6 or 7 is each assigned to these handles 4 and 5 in the longitudinal extension of the hand levers 2 and 3, respectively. The first hand lever 2 has a flat first base plate 8 between its handle 4 and its clamping jaw 6, while a second flat base plate 9 is provided between the second handle 5 and the clamping jaw 7 of the second hand lever 3. These two base plates 8 and 9 may be designed with different thicknesses. In the present embodiment variant, both base plates 8, 9 have approximately the same thickness, however. In the mounted state of the two hand levers 2 and 3 shown in FIG. 1 and in the “closed” pivoting position of the two hand levers 2 and 3 shown, the first base plate 8 and the second base plate 9 lie flat on top of one another.

A pivot pin 10, via which the base plates 8, 9 and thus the two hand levers 2 and 3 are connected to one another in relation to one another and pivotably, is provided in the area of these two base plates 8 and 9. In the embodiment variant shown, the pivot pin 10 is connected to the first base plate 8 in a fixed manner and passes through a corresponding bearing bore 11 of second base plate 9, whereby this bearing bore 11 can be seen in FIG. 1 only as a suggestion. Furthermore, it is apparent from FIG. 1 that the pivot pin 10 projects over the second base plate 9 outwardly and has a radially expanded ring flange 12 in its free end area.

In the closed pivoting position of the two hand levers 2 and 3 shown in FIG. 1, the second base plate 9 extends behind two locking webs 13 and 14, facing one another in relation to the pivot pin 10, which have a distance to the first base plate 8 corresponding to the thickness of the second base plate 9 and run in parallel to same. The first locking web 13 is arranged in the transition area from the first base plate 8 to the first handle 4 and the second locking web 14 is arranged in the transition area from the first base plate 8 to the first clamping jaw 6. It can be seen that the second base plate 9 in this closed pivoting position is accommodated with little play between the two locking webs 13, 14 and the first base plate 8. It is guaranteed by this design that the second base plate 9 cannot be raised from the first base plate 8 in the direction of the pivot pin 10 in the “normal” operation of the hand tool 1, such that the handles 4 and 5 as well as the two clamping jaws 6 and 7 are pivotable in relation to one another lying in an essentially common plane. To be able to clean the hand tool 1 as optimally as possible, it is necessary to make especially the two contact surfaces, facing one another, of the base plates 8 and 9 “lying” on top of one another accessible. For this, the hand tool 1 with its hand levers 2 and 3 is brought from the closed pivoting position shown in FIG. 1 into an open pivoting position.

For this, FIG. 2 shows an enlarged, perspective view of the hand tool 1, whereby the two handles 4 and 5 of the first and second hand levers 2 and 3 are each shown reduced. In FIG. 2, the first and second hand levers 2 and 3 are located in an “open” pivoting position, in which the first and second clamping jaws 6 and 7 with their clamping surfaces 15 and 16 facing one another have a distance from one another. The arrangement of pivot pin 10 in the two base plates 8 and 9 is selected, such that the flat clamping surfaces 15 and 16 of the two clamping jaws 6 and 7 can be pressed against one another in case of corresponding actuation of the handles 4 and 5.

In the “open” pivoting position of the two hand levers 2 and 3 shown in FIG. 2, the two clamping surfaces 15 and 16 of the clamping jaws 6 and 7 are already freely accessible, such that these can be acted upon by a corresponding cleaning liquid. The second base plate 9 is pivoted in relation to the first base plate 8, such that the first “contact surface” 17 of the first base plate 8 and the second “contact surface” 18 of the second base plate 9 are partly mutually covered. As a result, the “still” covered areas of the contact surfaces 17 and 18 cannot be acted upon by the cleaning liquid or at least not sufficiently.

To be able to now also make these areas of the contact surfaces 17 and 18 of the first base plate 8 and of the second base plate 9 accessible, the pivot pin 10 is provided with its radially expanded ring flange 12. As already mentioned in FIG. 1, the pivot pin 10 passes through the bearing bore 11 of the second base plate 9, such that the ring flange has a certain distance to the contact surface 17 of the first base plate 8 and especially also to the “outer” surface 19 of the second base plate 9.

Furthermore, it is apparent from FIG. 2 that, in the open pivoting position, the second base plate 9 is not in contact with the two locking webs 13 and 14. Thus, it is now possible, because of the distance of the radially expanded ring flange 12 from the surface 19 of the second base plate 9, to raise the second hand lever 3 in the direction of arrow 20 or in the direction of the pivot pin 10 from the contact surface 17 of the first base plate 8.

FIG. 3 shows such a raised state. It can be seen from FIG. 3 that the second base plate 9 is raised from the first base plate 8, whereby, in the embodiment variant shown, the pivot pin 10 with its radially expanded ring flange 12 is accommodated in a radially expanded hole expansion 21 of the bearing bore, which cannot be further seen in FIG. 3. The hole expansion 21 is embedded as a type of recess in surface 19 of the second base plate 9. Thus, it can be seen from FIG. 3 that the pivot pin 10 with its lower end area protruding towards the first base plate 8 from the second base plate 9, as well as the two contact surfaces 17 and 18 of the base plates 8 and 9 are freely accessible.

Due to the radially expanded design of the ring flange 12 of the pivot pin 10, a further “pushing apart” of the two base plates 8 and 9 is prevented, such that the hand tool 1 in the position shown in FIG. 3 can be cleaned without the hand tool 1 with its hand levers 2 and 3 being capable of being inadvertently separated from one another.

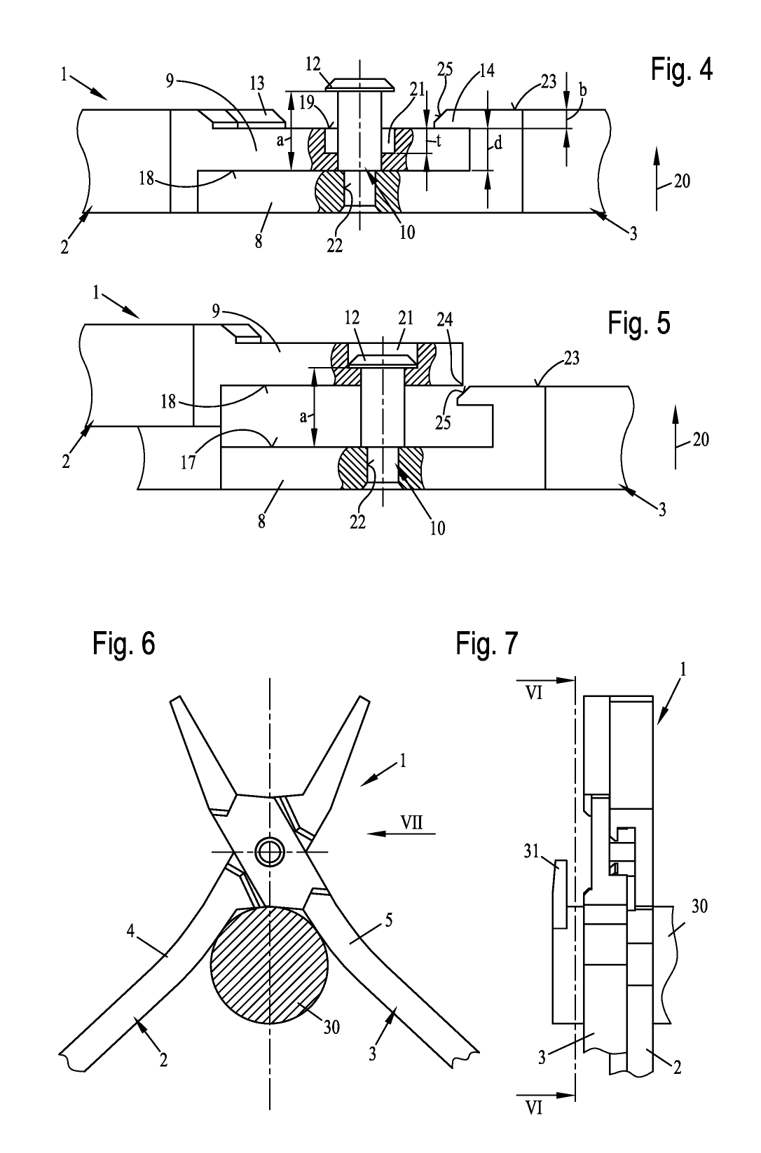

For this, FIG. 4 shows a partial lateral view of the hand tool 1 in the area of the two base plates 8 and 9, which are shown in a partial section in FIG. 4 in the area of the pivot pin 10. The two hand levers 2 and 3, which can be seen as a suggestion, are located in their closed pivoting position, as this can be seen in the perspective view from FIG. 1.

It is apparent from FIG. 4 that the second base plate 9 extends behind the two locking webs 13 and 14, such that a raising in the direction of arrow 20 in this operating position shown in FIG. 4 is certainly prevented.

The pivot pin 10 is inserted tightly into a mounting hole 22 of the first base plate 8 and it passes through the second base plate 9 vertically upwards. The pivot pin 10 may be, for example, welded, soldered, bonded or even screwed into the mounting hole 22 in a fixed manner. The “axial” length of the pivot pin 10 is selected in such a way that the distance a between the first base plate 8 and the ring flange 12 corresponds approximately to twice the thickness d of the second base plate 9. In the embodiment variant shown of FIG. 4, the ring flange 12 has a distance, which is smaller than the thickness d of the second base plate 9, to the surface 19 of the second base plate 9 in the shown, closed pivoting position of the two hand levers 2 and 3.

The hole expansion 21, designed as a recess, which is likewise apparent in FIG. 4, is designed as being somewhat larger in its “depth” t than the thickness b of the two locking webs 13 and 14. It is consequently achieved that, when the second base plate 9 is moved in the direction of the arrow 20 along the pivot pin 10, the lower contact surface 18 of the second base plate 9 lies approximately in the area of the surface 23 of the locking web 14 in the completely pushed-apart state of the two base plates 8 and 9, as this can be seen from FIG. 5. By means of a relative pivoting movement of the second base plate 9 to the first base plate 8, the contact surface 18 can be brought into contact with this surface 23, such that this acts as a stop face and thus it is prevented that the two base plates 8 and 9 are unintentionally pushed together again. The stop face 23 may have an increased coefficient of friction or be provided with locking teeth and/or even with locking means for “stopping” the two base plates 8, 9. To be able to bring the two hand levers 2, 3 into this locking position, an obliquely running guiding edge 25 may be provided at the “front edge” of the locking web 14. Because of this “locking,” it is also possible to rest, for example, to hang freely the hand tool 1 in any other position in the jet area of a cleaning machine.

This pushed-apart position of the two base plates 8 and 9 can be seen in FIG. 5, whereby in FIG. 5 the corresponding reference numbers are entered. In FIG. 5, the two hand levers 2 and 3 are “not yet” located in their locking position. In this raised position of the two base plates 8 and 9 shown in FIG. 5, their contact surfaces 17 and 18 as well as the part of the pivot pin 10 located between the two base plates 8 and 9 are freely accessible. Thus, these parts can basically be cleaned by means of a machine in a liquid-jet-generating cleaning machine. By means of the radially protruding ring flange of the pivot pin 10, a “falling apart” of the two hand levers 2 and 3 due to the action of the jets is certainly prevented.

To keep the two hand levers 2 and 3 in their “cleaning position,” the hand tool 1 may also be placed onto a holding device 30, for example, with its two handles 4 and 5, so that an inadvertent closing of the two hand levers 2 and 3 during the cleaning process is likewise certainly ruled out. For this, FIGS. 6 and 7 show in an exemplary manner such a contact of the hand tool 1 with such a holding device 30. It can be seen from FIG. 7 that this holding device 30 may have a vertically upwards standing locking web 31 in its free end area. This holding device 30 can be arranged in a cleaning chamber of a cleaning machine, not shown further.

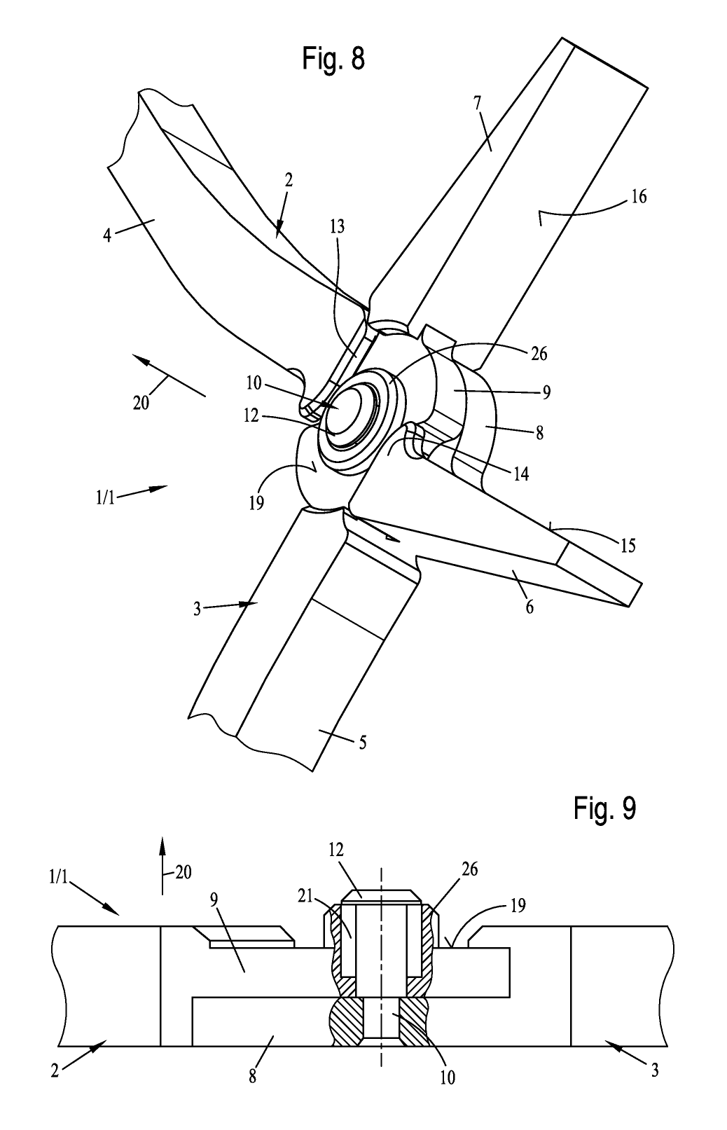

FIGS. 8 and 9 show views of a second embodiment variant of a hand tool 1/1, which is likewise formed from two hand levers 2 and 3 pivotable against one another. The two handles 4 and 5 of the two hand levers 2 and 3 are shown reduced in FIG. 8. Likewise, the two hand levers 2 and 3 each have a tool in the form of a clamping jaw 6 and 7, which are provided with corresponding clamping surfaces 15 and 16. The hand tool 1/1 from FIG. 8 is located in the same pivoting position as this is shown for the hand tool 1 shown in FIG. 2 for the embodiment variant 1 there. Furthermore, the embodiment variant of the hand tool 1/1 from FIG. 8 also has a first base plate 8 and a second base plate 9. In the closed state of the hand levers 2 and 3, the second base plate 9 likewise extends behind two locking webs 13 and 14, as this is shown for the embodiment variant of the hand tool 1/1 of FIG. 1.

The embodiment variant of the hand tool 1/1 of FIG. 8 differs from the embodiment variant 1 of FIG. 1 in that the two base plates 8 and 9 each have a basic structure designed like a circular arc. Otherwise, it is functionally identical to the embodiment variant 1 of FIGS. 1 through 5, so that in the pivoting position of the two hand levers 2 and 3 to one another shown in FIG. 8, the second base plate 9 can likewise be raised from the first base plate 8 in the direction of arrow 20.

The area surrounding the ring flange 12, of the pivot pin 10 that can be seen only as a suggestion in FIG. 8, has a circular ring web 26 protruding over the outer surface 19 of the second base plate 9.

This ring web 26 is designed in its height in such a way that the ring flange 12 of the pivot pin 10 (see FIG. 9) is at least partly accommodated by the hole expansion 21 continuing up to into the ring web 26. The risk of injury, for example, by pinching a finger, is considerably reduced due to this ring web 26, especially when moving the second base plate 9 in relation to the first base plate 8 in the direction of arrow 20.

While specific embodiments of the invention have been shown and described in detail to illustrate the application of the principles of the invention, it will be understood that the invention may be embodied otherwise without departing from such principles.

What is claimed is:

1. A scissor-like or forceps-like medical hand tool comprising:
a first hand lever with a first handle, a first tool part and a flat first base plate in an area between the first handle and the first tool;
a second hand lever with a second handle, a second tool part and a flat second base plate in an area between the second handle and the second tool;
a pivot pin connecting the first and second hand lever to one another pivotably, the pivot pin being arranged fixed in the first base plate and being pivotably accommodated in a bearing bore provided in the second base plate, the pivot pin having a portion passing through the second base plate and a radially expanded ring flange at an end area, the radially expanded ring flange being at a distance from the first base plate that corresponds to twice a thickness of the second base plate or that is greater than twice the thickness of the second base plate, such that the second hand lever can be moved, in an open pivoting position, relative to the first hand lever along the pivot pin into a position, in which contact surfaces of the flat first base plate and the flat second base plate, which face one another, are exposed.

2. A hand tool in accordance with claim 1, wherein the bearing bore of the second base plate of the second hand lever, for mounting the pivot pin, is provided on an outside with a radially expanded hole expansion accommodating the ring flange of the pivot pin.

3. A hand tool in accordance with claim 1, wherein:
the first hand lever in the area of the first tool has a stop face protruding on an outside in a direction of the pivot pin and lying in the pivoting area of the second base plate of the second hand lever; and
the second base plate is supported with an edge area in a position at the stop face, raised from the first base plate.

4. A hand tool in accordance with claim 3, wherein the stop face lies in an area of a plane, in which the inner contact surface of the second base plate of the second hand lever, facing the first base plate, is located, when the second base plate is located on the pivot pin in its position removed farthest from the first base plate of the first hand lever.

5. A hand tool in accordance with claim 3, wherein the stop face is provided with an oblique guiding edge.

6. A hand tool in accordance with claim 1, wherein the first base plate and the second base plate have an at least approximately congruent surface shape and have essentially a same thickness.

7. A hand tool in accordance with claim 2, wherein:
in an area surrounding the hole expansion, a circular ring web, protruding in a direction of the pivot pin, is provided, in which the hole expansion continues; and
the ring web ends in an area of the ring flange of the pivot pin with the first base plate and the second base plate lying on top of one another.

8. A medical hand tool comprising:
a first hand lever with a first handle, a first tool part and a flat first base plate in an area between the first handle and the first tool;
a second hand lever with a second handle, a second tool part and a flat second base plate in an area between the second handle and the second tool;
a pivot pin pivotably connecting the first and second hand lever to one another, the pivot pin being fixed to the first base plate and having an extending portion extending from the first base plate to at an end having a radially expanded ring flange, the second base plate having a bearing bore through which said extending portion passes, the radially expanded ring flange being spaced from the first base plate by a distance that at least corresponds to twice a thickness of the second base plate, whereby the second hand lever can be moved, in an open pivoting position, relative to the first hand lever along the pivot pin into a position, in which contact surfaces of the flat first base plate and the flat second base plate, which face one another, are exposed.

9. A hand tool in accordance with claim 8, wherein the bearing bore of the second base plate of the second hand lever, for mounting the pivot pin, is provided on an outside with a radially expanded hole expansion accommodating the ring flange of the pivot pin.

10. A hand tool in accordance with claim 8, wherein:
the first hand lever in the area of the first tool has a stop face protruding on an outside in a direction of the pivot pin and lying in the pivoting area of the second base plate of the second hand lever; and
the second base plate is supported with an edge area in a position at the stop face, raised from the first base plate.

11. A hand tool in accordance with claim 10, wherein the stop face lies in an area of a plane, in which the inner contact surface of the second base plate of the second hand lever, facing the first base plate, is located, when the second base plate is located on the pivot pin in its position removed farthest from the first base plate of the first hand lever.

12. A hand tool in accordance with claim 10, wherein the stop face is provided with an oblique guiding edge.

13. A hand tool in accordance with claim 8, wherein the first base plate and the second base plate have an at least approximately congruent surface shape and have essentially a same thickness.

14. A hand tool in accordance with claim 9, wherein:
in an area surrounding the hole expansion, a circular ring web, protruding in a direction of the pivot pin, is provided, in which the hole expansion continues; and
the ring web ends in an area of the ring flange of the pivot pin with the first base plate and the second base plate lying on top of one another.
